(19) United States
(12) Patent Application Publication
(10) Pub. No.: US 2025/0271372 A1
(43) Pub. Date: Aug. 28, 2025

(54) A Sensing Device for Detecting Analytes Using a Base Material Having a Polymer Material Thereon, as Well as a Method for Manufacturing Such Sensing Device

(71) Applicant: Universiteit Maastricht, Maastricht (NL)

(72) Inventors: Hanne Dilien, Maastricht (NL); Kasper Brecht Leander Eersels, Maastricht (NL); Bart Robert Nicolaas Van Grinsven, Maastricht (NL); Thomas Jan Cleij, Maastricht (NL); Rocio Arreguin Campos, Maastricht (NL)

(21) Appl. No.: 18/847,935

(22) PCT Filed: Mar. 20, 2023
(86) PCT No.: PCT/NL2023/050144

§ 371 Date: Sep. 17, 2024

(30) Foreign Application Priority Data
Mar. 18, 2022 (NL) 2031326

(51) Int. Cl. G01N 25/18

(52) U.S. Cl. CPC G01N 25/18

(57) ABSTRACT

A sensing device for detecting an analyte is proposed, the device comprising a base material coated with an assay polymer, the assay polymer formulated to bind to the analyte, wherein a heat transfer property of the assay polymer varies responsive to an amount of the analyte bound thereto, wherein the assay polymer comprises a surface imprinted polymer imprinted through sedimentation and enriched with an additive having a thermal conductivity higher than the thermal conductivity of the surface imprinted polymer.

### FIELD

The present disclosure relates to sensing devices and methods of detecting analytes using a base material having a polymer material thereon.

### BACKGROUND

A sensing device for detecting an analyte comprising a base material coated with an assay polymer, the assay polymer formulated to bind to the analyte, wherein a heat transfer property of the assay polymer varies responsive to an amount of the analyte bound thereto is, for example, disclosed in International Patent Publication No. WO2017178081A1.

Such known sensing device implements a versatile inexpensive thermal readout technology based on the heat-transfer method (HTM) and/or the thermal wave transport analysis (TWTA) and is a prime example of the development of alternative detection technologies allowing fast, cost-effective and accurate detection of bacteria along the food supply chain over the last decades. The techniques of HTM and/or TWTA rely on the measurement of the changes in the thermodynamic properties of the polymer that derive when the binds to the synthetic receptor.

When using HTM/TWTA for bacteria detection, the sensitivity of the known sensing device is limited directly influenced by the two components of the device: the synthetic recognition element and thermal readout platform.

Accordingly, it is a goal of the present disclosure to provide an improved sensing device with an improved sensitivity, allowing

### SUMMARY

According to a first example of the disclosure, a sensing device is proposed for detecting an analyte comprising a base material coated with an assay polymer, the assay polymer formulated to bind to the analyte, wherein a heat transfer property of the assay polymer varies responsive to an amount of the analyte bound thereto, wherein the assay polymer comprises a surface imprinted polymer imprinted through sedimentation and enriched with an additive having a thermal conductivity higher than the thermal conductivity of the surface imprinted polymer.

In a preferred example, the additive is a carbon based additive, and in particular the carbon based additive is selected from the group consisting of but not limited to diamond dust, graphene oxide, carbon nanotubes.

Preferably, a sensing device according to the disclosure with an improved sensitivity is obtained the polymer is selected from the group consisting of but not limited to silicone based polymers, e.g. polydimethylsiloxane.

According to the disclosure, the sensing device may comprise a base material over the base material, wherein the assay polymer is secured to a surface of the base material and wherein the base material is selected from the group consisting of aluminum, glass, steel, copper, gold, quartz, and ceramic materials. This results in an improved sensing platform with rigid construction.

In a preferred example, the sensing device may comprise a processor in electrical contact with the thermocouple, the processor programmed to calculate an amount of the analyte bound to the assay polymer. Accordingly, the processor may be programmed to calculate a concentration of the analyte in a liquid in contact with assay polymer based at least in part on the amount of the analyte bound to the assay polymer.

Alternatively, the processor may be programmed to detect a phase shift between a thermal wave at a heat transfer element and an attenuated thermal wave at the base material.

In another advantageous example according to the disclosure, the processor may be programmed to calculate the concentration of the analyte in the liquid based at least in part on a difference in amplitude between the thermal wave at the heat transfer element and the attenuated thermal wave at the base material.

In particular, the assay polymer may be applied or provided over and in contact with the base material, thus further enhancing the overall sensitivity of the sensing device. In particular, the assay polymer may surround the base material.

The sensing device according to the disclosure the base material is or forms part of a thermo-sensing element.

The disclosure also pertains to a method forming a sensing device for detecting an analyte, wherein the sensing device comprises a base material coated with an assay polymer, the assay polymer formulated to bind to the analyte, such that a heat transfer property of the assay polymer varies responsive to an amount of the analyte bound thereto, the method comprising at least the steps of:
i) providing a polymer based resin;
ii) dispersing in the polymer based resin an additive having a thermal conductivity higher than the thermal conductivity of the polymer based resin;
iii) applying a layer consisting of the polymer based resin with the dispersed additive on a base material;
iv) applying an analyte containing solution on the layer consisting of the polymer based resin with the dispersed additive;
v) allowing sedimentation of the analytes from the analyte containing solution on the surface of the layer consisting of the polymer based resin with the dispersed additive;
vi) removing residual solution and sedimented analytes from the layer consisting of the polymer based resin with the dispersed additive, thereby forming the assay polymer.

In a preferred example, the step ii) may comprise the sub steps of:
ii-1) cooling the polymer based resin during the dispersing step using ice-water; and
ii-2) subsequently mixing the polymer based resin with the dispersed additive with a curing agent at a ratio of 10:1 (w/w).

Additionally, in an example of the method according to the disclosure, step iii) may comprise:
iii-1) spin coating the base material with 125-175 μL, in particular 150 μL silicone based resin with the dispersed additive for at least 45-90 sec, in particular for 60 sec, at 5000 rpm. In particular, step iv) is preceded by the sub step of:
iii-2) pre-curing the spin coated layer of polymer based resin with the dispersed additive at 60-75° C. in particular at 65° C. for at least 5-25 minutes, in particular for 20 minutes, and more in particular for 10 minutes.

Furthermore, step v) may comprise the sub step of:
v-1) curing the layer consisting of the polymer based resin with the dispersed additive containing the analyte containing solution on the surface thereof at 60-75° C. in particular at 65° C. for at least 2-4 hours, in particular for 3 hours.

In a further example, the step vi) may comprise the sub step of:
vi-1) removing the residual solution and the sedimented analytes using deionized water and a 3% sodium dodecyl sulfate solution.

In order to acquire the sensing device according to the disclosure step iv) is followed by the step of:
vii) coating the base material with the assay polymer.

In the method according to the disclosure, it is preferred that the additive is a carbon based additive, in particular the carbon based additive is selected from the group consisting of but not limited to diamond dust, graphene oxide, carbon nanotubes. Additionally, it is preferred that the polymer is selected from the group consisting of but not limited to silicone based polymers, e.g. polydimethylsiloxane.

### BRIEF DESCRIPTION OF THE DRAWINGS

FIG. 1A a schematic representation of the interfacial surface-imprinting process of a sensor according to the disclosure.

FIG. 1B depicts brightfield microscopy imagery of E. coli safranin stained on imprinted polymer.

FIG. 1C depicts brightfield microscopy of empty bacteria cavities on polymer and FIG. 1C depicts the scanning electron microscopy of bacteria imprints.

FIG. 2A schematically depicts a representation of the thermal recognition of E. coli.

FIG. 2B depicts a fluorescence microscopy image visually depicting the rebinding of stained target to the surface of the polymer (PDMS-GO 0.01%) after flushing with PBS unbound cells.

FIGS. 2C-2D shows a real-time temperature response of the sensor employing surface-imprinted PDMS, PDMS-GO (0.01%) receptors (FIG. 2C) as well as the non-imprinted PDMS layers; SIP means Surface Imprinted Polymer (FIG. 2D).

FIG. 3 depicts the effect size curves for selectivity of the receptors (PDMS-GO 0.01%) against S. aureus experiment, with employed bacteria concentrations employed of 0, 1×102, 1×103, 5×104, 1×106 and 1×107 CFU/mL. Average values for each concentration are obtained from multiple measurements and error bars are calculated making use of the noise of the signal of the sensor.

FIG. 4 depicts the effect size curve for detection of E. coli in juice, with employed bacteria concentrations of 0, 1×102, 5×103, 1×104, 5×106 and 1×107 CFU/mL. Average values for each concentration are obtained from multiple measurements and error bars are calculated making use of the noise of the signal of the sensor according to the disclosure.

### DETAILED DESCRIPTION OF THE DISCLOSURE

For a proper understanding of the disclosure, in the detailed description below corresponding elements or parts of the disclosure will be denoted with identical reference numerals in the drawings.

This disclosure presents an imprinted polymer-based thermal biomimetic sensor for the detection of target analytes, such as bacteria, such as Escherichia coli. The disclosure also pertains to a novel and facile analyte bacteria imprinting manufacturing method or protocol for polydimethylsiloxane (PDMS) films and these receptor layers are functionalized with graphene oxide (GO) in order to improve the overall sensitivity of the sensor device. Upon the recognition and binding of the target to the densely-imprinted polymers, a concentration-dependent measurable change in temperature is observed. The limit of detection attained for the sensor employing PDMS-GO imprints was 80±10 CFU/mL, a full order lower than neat PDMS imprints (670±140 CFU/mL), illustrating the beneficial effect of the dopant on the thermodynamical properties of the interfacial layer. A parallel benchmarking of the thermal sensor with a commercial impedance analyzer was performed in order to prove the possibility of using the developed PDMS-GO receptors with multiple readout platforms. Moreover, S. aureus was employed as an analogue species for the assessment of the selectivity of the device. Finally, due to the potential that this biomimetic platform possesses a low-cost, rapid and on-site tool for monitoring E. coli contamination in food safety applications, spiked fruit juice is analyzed as real sample. Reproducible and sensitive results fulfill the limit requirements of the applicable European microbiological regulation.

Bacteria are ubiquitous microorganisms. While the majority of them are involved in beneficial interactions with the environment, animals and humans, certain microbes possess the potential of causing infectious diseases. Escherichia coli, for instance, is a bacterium typically found in the human intestinal tract. Whereas most E. coli strains will not cause any damage, some have been identified multiple times as the origin of foodborne illness outbreaks with global repercussions at public health and economical levels as detailed by the World Health Organization in 2018.

In order to avoid this, food processors employ routine bacteria detection methods such as plate counting and molecular-based technologies (e.g. immunoassays) to monitor the microbial contents of their products. Although these procedures are highly selective and extremely sensitive, they can be time-consuming, laborious and, in some cases, costly. Therefore, a lot of research efforts have been focused on the development of alternative detection technologies that allow fast, cost-effective and accurate detection of bacteria along the food supply chain over the last decades.

Biosensors for bacteria quantification have been developed in diverse fields where rapid, on-site testing is needed, such as medical diagnosis or environmental monitoring. In food safety, these devices have exhibited the potential to overcome inherent challenges of foodstuff analysis, namely complex matrices and attaining sensitivities that comply with the applicable microbiological criteria. Nonetheless, the usage of biological receptors in sensing platforms also holds some limitations including fragility, the requirement of carefully regulated conditions (pH, ionic strength, temperature, etc.) and limited shelf life. In order to fill in this gap, imprinted polymers as biomimetic alternatives have been recognized for their chemical stability and desirable affinities. The possibility of combining these synthetic receptors with a wide variety of transducing technologies (optical, electrochemical, mass-sensitive, thermal) has made possible already their use in the detection of food contaminants.

A versatile inexpensive thermal readout technology is the heat-transfer method (HTM), that has received increasing attention in the last few years. Combining HTM with imprinted polymers has proven to be a particularly valuable approach for the construction of sensors for the detection of a wide range of targets, including small molecules, human cells, and bacteria. The fundamentals of the HTM rely on the measurement of the changes in the thermodynamic properties of the polymer that derive when the binds to the synthetic receptor.

When the HTM was investigated for bacteria detection, the sensitivity of the device was identified as an improvement area. The limit of detection for Escherichia coli (104 CFU/mL) hindered the introduction of the sensor to diverse applications including its use in food safety management. This sensitivity is directly influenced by the two components of the device: the synthetic recognition element and thermal readout platform. On one hand, in order to attain a reproducible and appreciable binding behavior, the synthetic receptor has to be prepared considering a variety of experimental parameters such as material selection (functional monomers and ratios) as well as the polymer cell-imprinting technique.

In order to achieve the creation of cavities on the surface, specialized protocols such as the use of template stamps in micro-contact imprinting are required. This does not only introduce batch to batch variations but also hamper the scalability of imprint preparation. Moreover, on the transducer platform side, polymers are known for possessing low thermal conductivity (usually lower than 0.5 W/mK), which impacts in the noise of the device when translating the signal derived from the recognition event.

A modification on the heat-transfer method is based on the implementation of a planar meander element, which has resulted in low noise level in de device and therefore, a significant decrease in the detection limit for E. coli. Notwithstanding the fact that this improvement pushed forward the applicability of the sensor, making the thermal readout platform more specialized in terms of flow cell design and components represents a tradeoff with the simplicity, and therefore cost price, of the device. Similarly, stringently controlled advanced polymer imprinting techniques are more complex and require additional instrumentation for upscaling polymer synthesis.

According to the disclosure, a sensor design is proposed that aims to enhance sensitivity while maintaining the original simple thermal readout platform. This approach targets the aforementioned challenges of synthetic receptor preparation in terms of material selection and imprinting protocol. In order to avoid ab initio laborious imprinted polymer synthesis, the commercially available elastomer polydimethylsiloxane (PDMS), known for its moldability, chemical and mechanical robustness is proposed for its use as recognition element in the HTM biomimetic platform. This material has been cell and bacteria-imprinted employing micro-contact and roll-to-toll techniques, exhibiting the ability to recognize and sort these targets based on both morphology and chemical functionality.

Additionally, the disclosure presents a novel and simple surface imprinting protocol for PDMS that consists in the free assembly of the microorganism onto the surface of the polymer without the aid of a stamp. This approach enables scalability in the preparation of the receptor layers. Furthermore, in order to address the inherent low thermal conductivity of the synthetic receptor, in a further example of the disclosure, a functional additive is used in order to improve the response of the HTM transducer.

Carbon based additives (denoted with reference numeral 13 in the Figures), such as diamond dust and graphene oxide (GO) have been widely researched as filler for polymers. It has been observed that even a small loading of this carbon material has the ability of transferring its outstanding physicochemical properties to the composite. This attribute of GO has been already explored in imprinted polymers in combination with electrochemical readouts.

According to this disclosure, carbon based additives 13, such as graphene oxide flakes are used as an additive or filler for manufacturing imprinted PDMS layers 12 with the aim of obtaining a material with increased thermal conductivity, investigating the impact on the overall sensitivity of the sensor. Moreover, in order to benchmark with a commercial transducing platform, these results are compared in parallel with an impedance analyzer. Finally, the proposed sensor 10 is validated in fruit juice and correlated to the applicable legal limits established by the European Commission in order to explore the application of the proposed sensor in food safety.

Lysogeny broth (LB), CASO broth, safranin, sodium dodecyl sulfate (SDS), phosphate buffer saline (PBS) and anhydrous tetrahydrofuran (THF) were used as well as ethanol 70%, and a BacLight bacteria fluorescent stain. All reagents used had a minimum purity of 99.9%. Furthermore, E. coli ATCC8739 and Staphylococcus aureus (ATCC 6538) strains are used for imprinting purposes. As an example of the carbon based additive, graphene oxide (GO) flakes synthesized following an improved Hummer's method were used. All aqueous solutions were prepared with deionized water with a resistivity of 18.1 MΩ cm-1.

LB and CASO broths were prepared according to the standard protocols. 25 mL of LB and CASO were inoculated with a single colony of E. coli and S. aureus respectively (both denoted e.g. with reference numeral 50 in the Figures), and gently shaken at 120 rpm overnight at 37° C. Subsequently, 0.5 mL of the cultures were diluted in 4.5 mL of fresh broth and left for further growth for 2 h. Bacteria concentrations were calculated by measuring the OD600. The cultures were centrifuged at 3000 rpm for 5 min, and the obtained pellets were re-suspended in PBS. This washing procedure was repeated once and finally, the bacteria were diluted with sterile PBS to obtain the desired concentrations.

Graphene oxide flakes 13 were dispersed into a polydimethylsiloxane base resin 12 employing a Fisherbrand sonic dismembrator with a probe of 2 mm diameter. In order to avoid heat generation, the PDMS was kept in an ice-bath during the sonication process. Subsequently, the base 12 containing GO 13 was mixed with the curing agent following the ratio of 10:1 (w/w). The viscous mixture was employed for preparing a stock solution of 10% PDMS in tetrahydrofuran (w/w). A stock solution of neat PDMS was prepared following the same procedure except for the addition of GO.

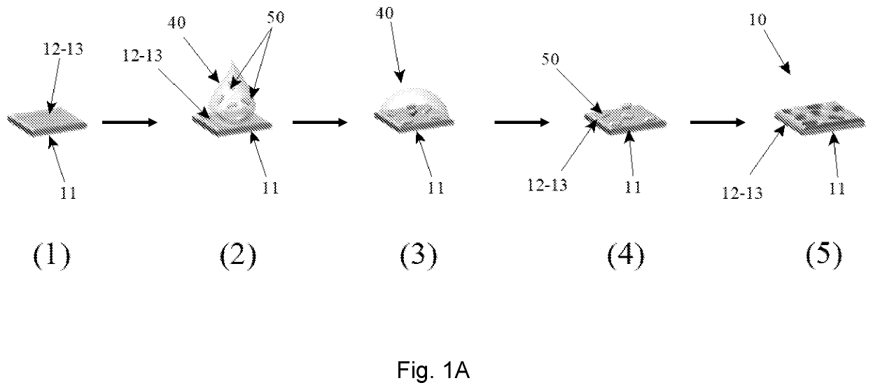

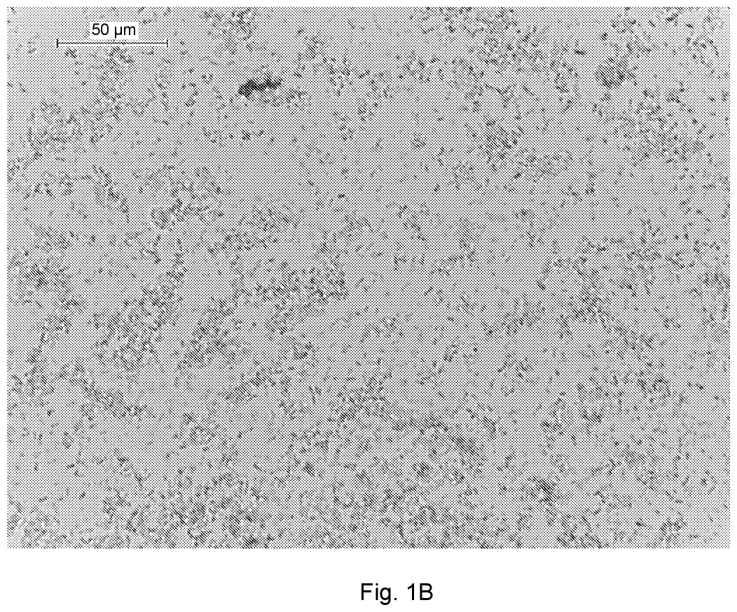

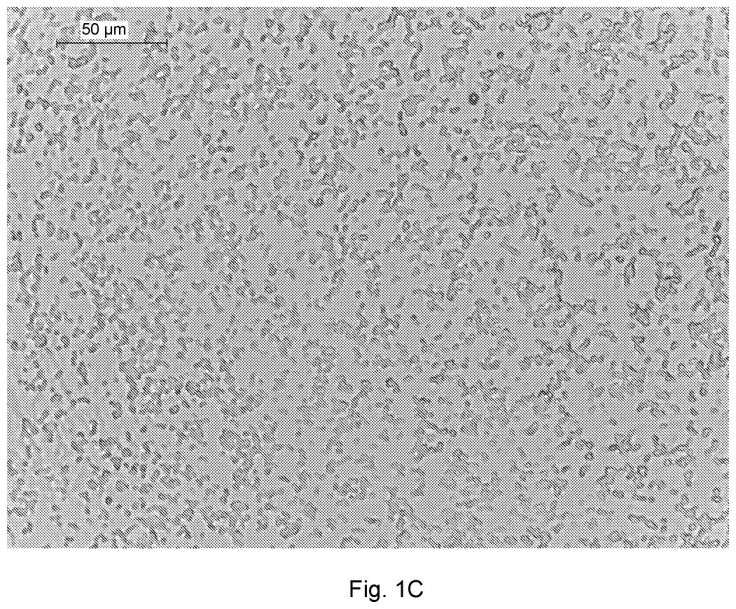

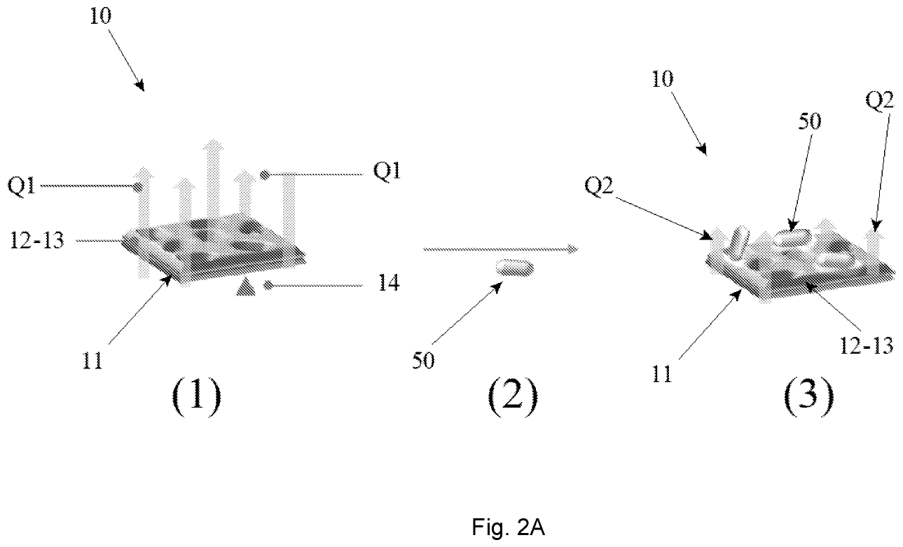

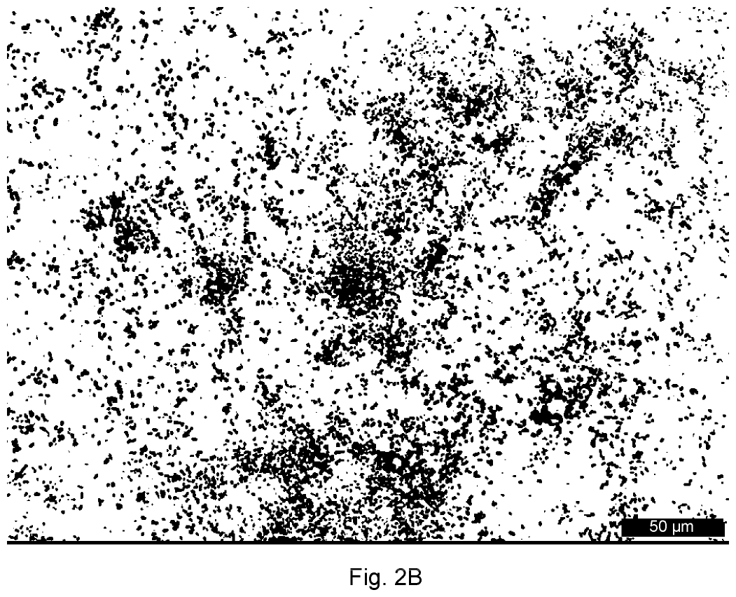

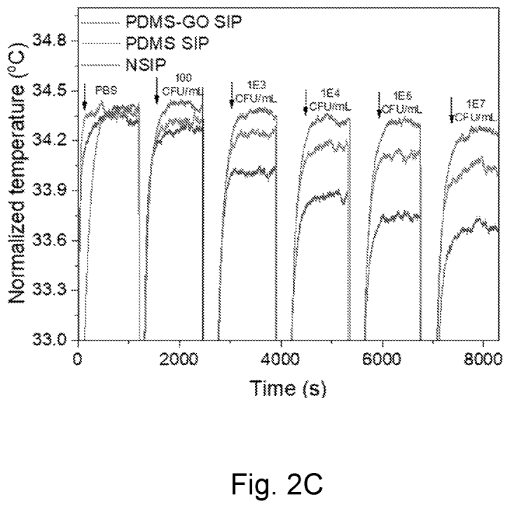

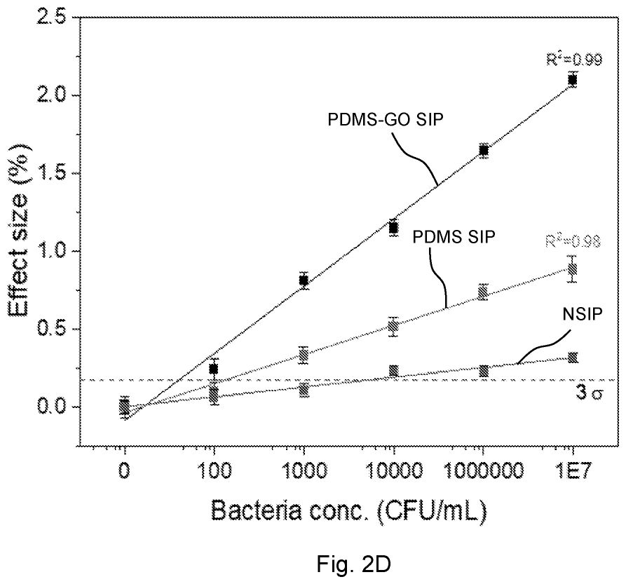

As examples of a base material 11, microscope glass slides and/or 1 cm2 aluminum chips were spin-coated for 60 s at 5000 rpm with 150 μL of the prepared PDMS and PDMS-GO stock solutions 12-13, see FIG. 1A-1. A pre-curing treatment for the resin was performed on the substrates at 65° C. for 10 minutes. Subsequently, 250 μL of bacteria solution (1×108 CFU/mL) (reference numeral 40-50 in the Figures) were applied onto the surface of the pre-cured films 12-13 (see FIG. 1A-2) and left for sedimentation for 20 minutes at room temperature (see FIG. 1A-3). While maintaining the bacteria solutions on the surface of polymers, the substrates Nov. 12, 2013 were placed in the oven at 65° C. for three hours in order to achieve full curing of the PDMS (see FIG. 1A-4). The substrates/films Nov. 12, 2013 were finally washed with deionized water in order to remove residual salts from PBS buffer followed by SDS 3% to detach the template from the PDMS leaving behind the imprint cavities, see FIG. 1A-5. Other base materials 11, which can also be used, are steel, copper, gold, quartz, and ceramic materials. The base material 11 can function as a thermo-sensing element.

Bright-field microscopy was performed on a LEICA DM 750 optical microscope. ImageJ 1.440 was employed to calculate the average surface coverage of cell imprints on the polymeric layers. The number of cell imprints per area unit was determined based on the individual counts of three different batch samples and three locations on each imprint. In order to facilitate the visualization of the bacteria, safranin was employed as staining solution. Fluorescence microscopy was performed on an Olympus BX53 microscope, see FIG. 1B. With the aim of visually confirming the rebinding of the targeted bacteria to the prepared imprints, E. coli was stained with fluorescent dye according to the standard protocol. Imprinted films were exposed to a solution of 1×108 stained bacteria mL-1 for 20 minutes in order to allow recognition of the target. After this time, the films were rinsed with PBS in order to remove non-bound bacteria from the surface (FIG. 1A-5). The films were then observed under the microscope.

Scanning electron microscopy was carried out at 2.0 eV, using an iridium coating. The prepared imprinted polymers were observed in order to confirm the presence of the cavities on the surface and to analyze their morphology, see FIG. 1C.

The setup for the HTM device is a known technique. Briefly, the surface-imprinted chips 10 were placed backside onto a copper block that performs as heat sink (reference numeral 14 in FIG. 2A-1). The block is then coupled to a PMMA (poly methyl methacrylate) flow cell by scaling the two pieces with an O-ring to avoid leakage. The contact area of the imprint is determined by the diameter of this ring (28 mm2), and the volume of the flow cell is 110 μL, which are introduced to the system using a tubing system via an automated syringe pump. The temperature of the heating block (T1=37° C.) is controlled by modifying the voltage over the power resistor using a proportional-integral-derivative (PID) software-based controller. The settings employed have been optimized in previous work (P=10, I=8, D=0). T1 and the temperature of the chamber (T2) were monitored by K-type thermocouples (TC direct) placed in the copper block and at 1 mm above the chip, respectively.

Impedance was measured with a MFIA impedance analyzer. For this purpose, a gold wire of 0.5 mm diameter was adapted into the flow cell as electrode at the same position from the bottom of the chamber as the thermocouple in the opposite side. Continuous frequency sweeps of 200 points were taken between 100 and 10000 Hz at a test signal of 300 mV. Before each experiment, PBS is introduced into the flow cell and the system is allowed to stabilize. After this stabilization period, 2 mL of the desired bacteria solution is injected at a controlled flow rate of 2 mL/min. The stabilization time employed for bacteria in the system was 20 min, and afterwards a solution of SDS (3%) followed by PBS are flushed into the flow cell at the previously mentioned flow rate with the purpose of removing the bacteria from the polymer layers.

The HTM setup monitors the temperature and thermal resistance (Rth) and the electrode the impedance at the solid-liquid interface simultaneously. Dose-response curves for HTM were obtained from temperature data as reported previously for chemo-sensing employing the HTM. As for impedance, curves were obtained from the absolute impedance values at a single frequency at which the corresponding phase angle is between 40 and 50 degrees, focusing on the double layer represented by an “R-C” circuit. In an example according to the disclosure, commercially available watermelon-strawberry juice was acquired and used as received. After being tested for the absence of microorganisms, it was spiked with bacteria to obtain the desired concentrations. No additional sample preparation was performed.

Visual assessment of the imprinted polymers as prepared in accordance with the method steps of the disclosure was performed using Brightfield microscopy with the aim of confirming the presence of bacteria cavities on the PDMS layers. FIG. 1A-5 depicts the imprinted polymer according to the disclosure right after its preparation, where the presence of safranin-stained bacteria on the films can be observed. The analyte presents a heterogeneous distribution, and the notorious agglomeration of E. coli 50 can be highlighted due to the imprinting technique employed, in which the bacteria freely assembles onto the semi-cured PDMS, See FIG. 1B. The density of template on the surface of the polymer was calculated as 21.7±4.8%, an enhanced coverage when comparing with the values obtained for micro-contact imprinting techniques (14.3±1.8%) in previous studies. Removal of the bacteria was done by washing the imprinted layers with SDS 3% and with water to remove residual salts from the imprinting process.

FIG. 1C shows the surface of the material with empty cavities. The observed pockets on the PDMS match E. coli in shape and size, which was further confirmed with scanning electron microscopy, where the characteristic rod-shape morphology of E. coli (1-3 μm) was clearly identified (FIG. 1C). The compilation of these results confirm that PDMS synthetic receptors for the analyte of interest Escherichia coli 50 were successfully prepared via interfacial imprinting.

The results as outlined in this application clearly illustrate that it is possibly to achieve a morphological imprint of the bacteria 50 on the surface of the PDMS layer 12-13. In a next step, the receptor layers were prepared on aluminum chips 11 in order to quantify the rebinding of the template in PBS using the HTM readout platform. With the device according to the disclosure the effect was investigated of a functional additive (GO) in the receptor layers coupled to HTM as a strategy to enhance the sensor's signal without adding complexity to the readout method. For this purpose, neat PDMS as well as PDMS-GO (0.01%) were prepared. Rebinding was tested on both layer types and compared to the behavior of non-imprinted layers. The selection of a small load of GO aims to maintain a fast and simple homogenization process for the filler. Microscope and SEM imaging of the graphene oxide functionalized layers can be found in supplementary information (as shown in FIG. 1).

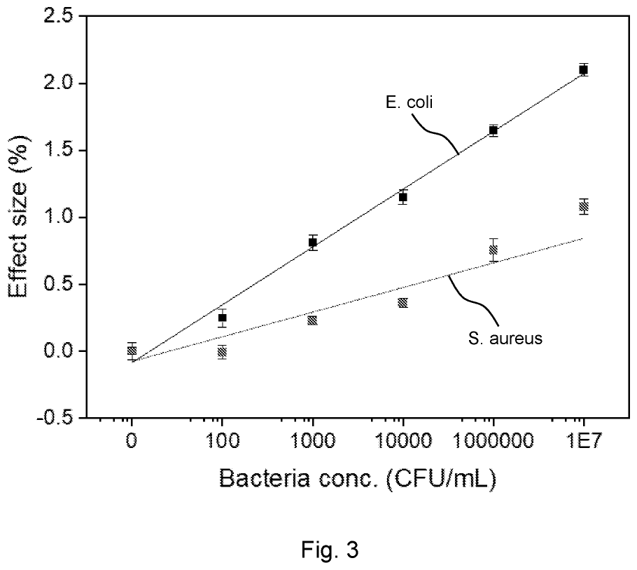

The experimental conditions for the HTM were kept constant in order to enable the direct comparison of the receptors. The flow cell system was filled with phosphate buffer as blank and the temperature of the copper heating element 14 was stabilized at 37° C. for 20 minutes (indicated with Q1 in FIG. 2A-1). Subsequently, the layers were exposed and incubated to an increasing concentration of E. coli suspensions 40-50 in PBS (see FIG. 2A-2) in order to allow the target to bind to the surface (FIG. 2A-3). In-between each incubation step, the films were rinsed in situ with SDS (3%) and buffer with the purpose of removing all the bound cells from the previous exposition before adding the next concentration.

The results of these experiments are shown in FIGS. 2C and 2D, where the sensor's real time response can be observed for the different tested layers. In the case of imprinted PDMS and PDMS-GO a clear diminishing of the flow cell's inside temperature (denoted in FIG. 2A-3 with Q2, with Q2<Q1 depicted in FIG. 2A-1) can be observed as the template concentration is increased, which is attributed to the augmentation in thermal resistance at the solid to liquid interface derived from the binding of the bacteria to the imprinted polymer (FIG. 2B). This can be confirmed when analyzing PDMS without cavities, for which only a small temperature response is observed due to non-specific interactions with the polymeric surface. Only when a high concentration of bacteria is infused into the system, a significant response is observed that barely exceeds the noise of the signal. Although both PDMS-based imprinted receptor layers exhibit this concentration-dependent trend of temperature change, it can be highlighted that the response obtained for the PDMS-GO receptors is more pronounced when comparing to neat PDMS. This can be directly linked to the presence of the carbon functional additive, which has been reported to confer thermal properties to polymers when used (in flakes, fibers, nanoparticles, etc.) in composites.

In order to link the temperature response obtained from the sensor to a visual confirmation of the template's rebinding, the target was stained with a labeling reagent and incubated for 20 minutes on the surface of the imprinted PDMS-GO. Subsequently, the layers were rinsed with buffer to remove unbound cells. In FIG. 2B, fluorescent microscope pictures of the synthetic receptor with and without the target depict the recognition event. Moreover, it can be highlighted that the heterogeneous and agglomerated distribution of rebound bacteria is in alignment with the observations made for the receptors.

In order to determine the limit of detection (LoD) of the biomimetic sensor when employing the different PDMS receptor layers, dose-response curves were constructed with the real-time obtained temperature data. Mean values for each incubation steps were obtained from 300 s intervals after stabilization of the signal. Subsequently, effect sizes were calculated employing the average temperature for each target concentration (t=c) with respect to the temperature of the baseline (t=0). The used formula was:

$$Effect size ( \% ) = \frac{Δ {T}_{t} ( t = c )}{{T}_{t} ( t = 0 )} \times 100$$

The effect size data of multiple experiments (in terms of chips and batches) was plotted against the normalized logarithmic bacteria concentrations and fit with OriginPro™ to an empirical linear function with the formula: y=a+b*x (FIG. 2D). The limits-of-detection were calculated as the lowest concentrations at which the effect size is higher than three times the averaged error collected for three data sets (green line, 3σ method). The LoD obtained for neat PDMS was 670±140 CFU/mL, which is in line with previously found values for bacteria-imprinted polyurethane layers. Furthermore, the limit for PDMS-GO is 80±10 CFU/mL, which confirms that the enhancement of the sensor's signal due to the presence of graphene oxide as additive, leads to an improvement in the overall sensitivity of the device.

The Heat Transfer Method platform has been proposed as a low-cost, rapid and user-friendly technology for biomimetic sensing. Due to the research that is being performed on its optimization, it is of value to compare its performance with other transducers. In order to benchmark the PDMS-GO/HTM biomimetic sensor with a commercial readout technology, real-time experiments following the protocol employed for the HTM were performed using impedance spectroscopy. The representative curves corresponding to the simultaneous monitoring of these readout techniques against time can be found in supplementary information (FIG. 2D).

As observed for HTM, the sensor exhibited a concentration-dependent drop in impedance that can be attributed to the fact that E. coli possess a negatively charged surface due to the presence of anionic groups on its outer membrane (e.g. carboxylates and phosphates). The drops in impedance and in temperature were employed for calculating the effect sizes (equation presented above) and the limits of detection of the sensor by fitting the response of the platforms to an empirical bacterial growth equation integrated in the OriginPro™ software package with the formula: y=a*(x−b)\hat{ }c.

LoD (3σ) and effect sizes are summarized on Table 1. These data illustrate that the impedimetric quantification of the template is one order of magnitude more sensitive when comparing to HTM. This can be attributed to the fact the accumulation of charge of E. coli will create a larger relative effect size in impedance as compared to the thermal resistance signal. Furthermore, it can be noticed that the effect sizes for the thermal readout when integrating it with impedance are around three times lower comparing to the results observed for the HTM by itself. This could be correlated to the aforementioned charge accumulation at the solid to liquid interface, which might create turbulence and hinder thermal transport from the receptor to the buffer liquid.

This phenomena leads to limits of detection that are higher and less reproducible, and therefore, suggests that whereas the PDMS-GO hereby presented are suitable for their use in both transducing platforms, better results can be expected when temperature and impedance are monitored separately. Comparing the data here to those obtained in the previous chapter, leads to the conclusion that HTM has demonstrated to be able to produce sensors with a similar sensitivity in comparison to impedance when combined with GO-doped PDMS layers.

TABLE 1
Simultaneous HTM/Impedance transducers integration.

| | |
|---|---|
| Impedance | HTM |

| | LOD | Effect size | LOD | Effect size |
|---|---|---|---|---|
| Batch | CFU/mL | % | CFU/mL | % |
| 1 | 26 | 6.2 | 120 | 0.67 |
| 2 | 30 | 7.3 | 700 | 0.44 |
| 3 | 30 | 15.1 | 321 | 0.64 |

Staphylococcus aureus was employed for assessing the selectivity of the PDMS-GO imprinted receptors. Increasing concentrations of this microorganism were infused into the experimental setup following the same conditions used for the targeted E. coli in order to compare the sensor's response. The results for this test are depicted in FIG. 3, where the effect size obtained for S. aureus could be linked to some extent of non-selective binding of this bacterium to the receptor layers. Nonetheless, the response observed for E. coli is roughly twice for all the concentrations for which the sensor was tested when comparing to S. aureus.

This difference relies on the fundamentals of target recognition of imprinted polymers. The imprinted E. coli is a rod shaped gram-negative bacteria, whereas S. aureus is a spherical gram-positive microorganism. The inherent distinct morphologies and membrane compositions result in a signal that derives from the interaction of the target with the receptor and the matching with the geometry of the cavities. In addition, previous research has shown that the selectivity attributed to imprinted PDMS layers, stems predominantly from the interaction of functional groups between the template species and the micro cavities on the surface of the imprinted layers. Taking this into consideration, selectivity of the receptors could be enhanced chemically by modification of the PDMS in order to tune its affinity to the target.

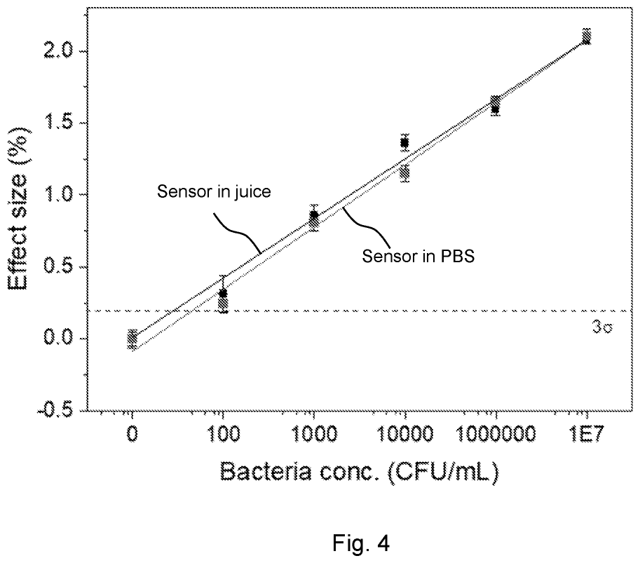

Spiked juice samples were analyzed with the PDMS-GO sensor and its performance was compared to the experiments in buffer summarized in previous chapter. The corresponding effect size curves obtained from multiple experiments are shown in FIG. 4, where it can be remarked the similarity of the device's performance in both liquids. The limit of detection calculated for the food sample (70±8 CFU/mL) is similar to the value obtained for PBS bacteria suspensions.

For this type of food sample (ready-to-drink fruit juices), the European Commission Regulation (EC) 1441/2007 on microbiological criteria for foodstuffs determines a legal limit of 100-1000 CFU/g for Escherichia coli (European Union, 2007). The results for the sensor hereby presented prove bacterial contamination in these type of juices as its linear range falls within these established values. In addition, according to the mentioned document, our sensor would also meet the limit specifications for the analysis of other food products including meat preparations (500-5000 CFU/g), cheeses from heat-treated milk (100-1000 CFU/g), among others. The challenge for the latter matrices would be to move from liquid samples to follow the adequate sample preparation techniques for solid foodstuff. These results confirm that biomimetic sensing platform has undergone the necessary improvements for implementation into commercial food safety assessment processes by improving the sensitivity by two orders of magnitude.

In Table 2, a summary of other relevant biomimetic platforms researched for E. coli in food samples in the last decade is presented. It can be noticed that the sensor's performance, derived from the optimization of the receptor layer, is competitive with other thermal platforms and devices based on electrochemical, micro-gravimetrical and optical readout technologies.

TABLE 2
Comparison of recently developed biomimetic platform applied to food samples.

| | Sensor | LoD | |
|---|---|---|---|
| Reference | Polymer/imprinting protocol/Transducer | CFU/mL | Sample |
| (van Grinsven | Polyurethane/Micro-contact/HTM | 1 E4 | Buffer |
| et al., 2016) | | | |
| (Yilmaz et al., | Polymethylmethacrylate/ Micro-contact/SPR, | 1.5 E6, | Apple |
| 2015) | QCM | 3.7 E5 | juice |
| (Chen et al., | Polydopamine/Electrodeposition/Electrochemical | 8 | Water |
| 2017) | | | |
| (Cornelis et | Polyurethane/Micro-contact/modified HTM | 100 | Apple |
| al., 2019) | | | juice |
| (Arreguin- | Polyurethane-co-urea/Micro-contact/HTM | 1000 | Milk |
| Campos et al., | | | |
| 2021) | | | |
| Present work | PDMS-GO/Interfacial/HTM | 70 | Strawberry- |
| | | | watermelon |
| | | | juice |

The disclosure introduces a facile and novel bacteria imprinting measuring device and method protocol for PDMS as a strategy to improve the analytical performance of the original heat transfer method sensing technology. This technique does not only attain a dense coverage of cavities on the receptors but also excludes the need for a template stamp. In combination with the use of a commercial resin, this procedure adds to the scalability of the synthesis process when comparing to the state of the art imprinting techniques. Moreover, graphene oxide as an additive to the synthetic receptors has been investigated for the first time for the enhancement of the thermal transducer's signal, massively decreasing the limit of detection for Escherichia coli to 80±10 CFU/mL. This sensitivity is competitive to other thermal devices that have focused solely on the modification of the readout by implementing extra components, which require periodical calibration and add complexity to the device. The major advantage of the novel approach undertaken in this study is that the sensor's performance is enhanced while the simple, low-cost and user-friendly nature of the sensing technology is not sacrificed.

The performance of the proposed sensor was compared to a commercial impedance analyzer, obtaining similar sensitivity. Despite the fact that the developed receptors were assessed simultaneously for the two transducing technologies, the obtained data suggest that better results are expected when coupling the imprinted polymers to the separate readout platforms. Nonetheless, every sensitive electrochemical readout platform will benefit from some form of temperature control, while the sensor readout used in this case is not more complicated than the average temperature control unit in a commercial impedance analyzer.

Finally, the main objective of the research was to improve the technology for application in food safety management settings. The enhancement of the biomimetic thermal has demonstrated to enable end-users to faithfully determine the concentration of bacteria in commercial fruit juices within regulatory limits without any sample preparation. When adapting a suitable sample preparation protocols, different food matrices could be analyzed and the concept could also be extended towards medical diagnostics or environmental screening. The further investigation of the performance of this device could lead to its validation against the current standards of microbiological testing protocols.

1. A sensing device for detecting an analyte comprising a base material coated with an assay polymer, the assay polymer formulated to bind to the analyte, wherein a heat transfer property of the assay polymer varies responsive to an amount of the analyte bound thereto, wherein the assay polymer comprises a surface imprinted polymer imprinted through sedimentation and enriched with an additive having a thermal conductivity higher than the thermal conductivity of the surface imprinted polymer.

2. The sensing device of claim 1, wherein the additive is a carbon based additive.

3. The sensing device of claim 2, wherein the carbon based additive is selected from the group consisting of but not limited to diamond dust, graphene oxide, carbon nanotubes.

4. The sensing device of claim 1, wherein the surface imprinted polymer is selected from the group consisting of but not limited to silicone based polymers, e.g. polydimethylsiloxane.

5. The sensing device of claim 1, wherein the base material is selected from the group consisting of aluminum, glass, steel, copper, gold, quartz, and ceramic materials.

6. The sensing device of claim 1 further comprising a processor in electrical contact with the base material, the processor programmed to calculate an amount of the analyte bound to the assay polymer.

7. The sensing device of claim 6, wherein the processor is programmed to calculate a concentration of the analyte in a liquid in contact with assay polymer based at least in part on the amount of the analyte bound to the assay polymer.

8. The sensing device of claim 6, wherein the processor is programmed to detect a phase shift between a thermal wave at a heat transfer element and an attenuated thermal wave at the base material.

9. The sensing device of claim 6, wherein the processor is programmed to calculate the concentration of the analyte in the liquid based at least in part on a difference in amplitude between the thermal wave at the heat transfer element and the attenuated thermal wave at the base material.

10. The sensing device of claim 1, wherein the assay polymer is over and in contact with the base material.

11. The sensing device of claim 1, wherein the assay polymer surrounds the base material.

12. The sensing device of claim 1, wherein the base material is or forms part of a thermo-sensing element.

13. A method forming a sensing device for detecting an analyte, wherein the sensing device comprises a base material coated with an assay polymer, the assay polymer formulated to bind to the analyte, such that a heat transfer property of the assay polymer varies responsive to an amount of the analyte bound thereto, the method comprising at least the steps of:
i) providing a polymer based resin;
ii) dispersing in the polymer based resin an additive having a thermal conductivity higher than the thermal conductivity of the polymer based resin;
iii) applying a layer consisting of the polymer based resin with the dispersed additive on a base material;
iv) applying an analyte containing solution on the layer consisting of the polymer based resin with the dispersed additive;
v) allowing sedimentation of the analytes from the analyte containing solution on the surface of the layer consisting of the polymer based resin with the dispersed additive;
vi) removing residual solution and sedimented analytes from the layer consisting of the polymer based resin with the dispersed additive, thereby forming the assay polymer.

14. The method of claim 13, wherein step ii) comprises the sub steps of
ii-1) cooling the polymer based resin during the dispersing step using ice-water; and
ii-2) subsequently mixing the polymer based resin with the dispersed additive with a curing agent at a ratio of 10:1 (w/w).

15. The method of claim 13, wherein step v) comprises the sub step of
v-1) curing the layer consisting of the polymer based resin with the dispersed additive containing the analyte containing solution on the surface thereof at 60-75° C. in particular at 65° C. for at least 2-4 hours, in particular for 3 hours.

16. The method of claim 13, wherein step vi) comprises the sub step of
vi-1) removing the residual solution and the sedimented analytes using deionized water and a 3% sodium dodecyl sulfate solution.

17. The method of claim 13, wherein step iv) is followed by the step of:
vii) coating the base material with the assay polymer.

18. The method of claim 13, wherein the additive is a carbon based additive, in particular the carbon based additive is selected from the group consisting of but not limited to diamond dust, graphene oxide, carbon nanotubes.

19. The method of claim 13, wherein the polymer is selected from the group consisting of but not limited to silicone based polymers, e.g. polydimethylsiloxane.
